(19) United States
(12) Patent Application Publication
(10) Pub. No.: US 2025/0099360 A1
(43) Pub. Date: Mar. 27, 2025

(54) Skincare Formulation with Increased Stability, Delivery and Bioavailability of Vitamin C

(71) Applicant: Goodier Cosmetics, LLC, Dallas, TX (US)

(72) Inventors: Tatiana Kelly, Westlake, TX (US); Lee Vickers, Irving, TX (US)

(21) Appl. No.: 18/373,652

(22) Filed: Sep. 27, 2023

(51) Int. Cl. A61K 8/67; A61Q 19/00

(52) U.S. Cl. CPC A61K 8/676; A61Q 19/00

(57) ABSTRACT

A skincare formulation for improving the stability, delivery and bioavailability of vitamin C. The skincare formulation includes active vitamin C derivatives mixed into a carrier base. The active vitamin C derivatives include an effective amount of a hydrophilic vitamin C derivative, a lipophilic vitamin C derivative, and an amphiphilic vitamin C derivative, but exclude L-ascorbic acid and ascorbyl palmitate. A pH of the skincare formulation is greater than 3.5.

### TECHNICAL FIELD

Aspects of the present disclosure relate to a skincare formulation. More particularly, the present disclosure is directed to a skincare formulation with active vitamin C derivatives that can provide an increased bioavailability of vitamin C with reduced stability issues such as degradation that causes undesirable discoloration and malodor.

### BACKGROUND

Vitamin C, also known as L-ascorbic acid, is a water-soluble (i.e., hydrophilic) vitamin essential for human growth and development. For example, vitamin C is needed for biosynthesis of blood vessels, collagen, cartilage, and muscle. Vitamin C is also involved in protein metabolism and is a potent antioxidant that protects cells against injury from free radicals that can be formed during the breakdown of food and as a result of exposure to radiation, e.g., the sun and X-rays.

Humans are unable to synthesize vitamin C. A varied diet that includes citrus fruits, berries, and other fruits and vegetables can provide the daily recommended amount of vitamin C. However, the absorption of vitamin C in the digestive tract is regulated and limited. For example, some studies have shown that doses of vitamin C exceeding 1 gram per day are absorbed at a rate that is less than 50% and the remainder of the vitamin C remains unmetabolized and is excreted in the urine. In the body, the highest levels of vitamin C are present in leukocytes, eyes, adrenal glands, the pituitary gland, and the brain. Relatively low amounts of vitamin C are present in the skin.

### SUMMARY OF THE INVENTION

Aspects of the disclosure are directed to a skincare formulation that contains high concentrations of vitamin c compounds, which simultaneously increases the delivery and bioavailability of vitamin C in the skin while enhancing the stability of the vitamin c compounds in the formulation. The skincare formulation includes an effective amount of active vitamin c derivatives mixed into a carrier base. The active vitamin c derivatives include a hydrophilic vitamin C derivative, a lipophilic vitamin C derivative, and an amphiphilic vitamin C derivative, but excludes ascorbic acid and ascorbyl palmitate. The pH of the skincare formulation is greater than 3.5.

Other aspects, embodiments, and features of the invention will become apparent from the following detailed description of the invention.

### DETAILED DESCRIPTION

Oxidative stress in the skin, which can be a result of intrinsic factors such as cellular metabolism and/or extrinsic factors such as exposure to solar radiation and pollution, causes the formation of reactive oxygen species, also commonly referred to as free radicals. Free radicals are unstable molecules that can cause damage to nucleic acids, proteins, and cell membranes. For example, ultraviolet (UV)-induced free radicals can increase the production of metalloproteinases (MMPs), which degrade collagen, reduce collagen production, and increase abnormal elastin accumulation, all of which contribute to photoaging pigmentation, coarse texture, and wrinkles.

Vitamin C is a potent antioxidant that can neutralize oxidative stress by a process of electron transfer and/or donation. Additionally, vitamin C promotes collagen biosynthesis and replenishes vitamin E, a lipophilic antioxidant that also helps protect against oxidative stress. Vitamin C also inhibits the action of tyrosinase, an enzyme responsible for irregular melanin production that occurs with age, i.e., photoaging pigmentation.

Due to the relatively low amounts of dietary vitamin C available in the skin, efforts have been directed toward creating a skincare formulation capable of delivering vitamin C directly to the skin. However, vitamin C in the L-ascorbic acid form is unstable and historically formulations that included meaningful concentrations of L-ascorbic acid became discolored and tacky, and developed a malodor. Efforts to improve the stability of higher concentration L-ascorbic acid serums were made in subsequent skincare formulations by creating a formulation that had at least 10 wt % vitamin C and a low pH of <3.5. At such a low pH, L-ascorbic acid is un-ionized and more stable, but the acidic pH causes microstressing of the skin and damages the skin microbiome.

Aspects of the present disclosure recognize the need for a skincare formulation that includes active vitamin c derivatives capable of providing the benefits of vitamin C in the skin, but which have enhanced stability without the need for increased acidity. Additionally, aspects of the present disclosure recognize the need for the active vitamin c derivatives to be delivered to the skin at a high enough concentration to have a biologic effect, i.e., be present at an effective amount. These aspects of the present disclosure can be achieved by careful selection of the active vitamin c derivatives. For example, replacing L-ascorbic acid with one or more stable vitamin C derivatives can provide the desired stability without the need for the accompanying acidity. A pH between 3.5-7, and more specifically around a skin neutral pH between 4.0-6.0, and more specifically a pH of about 5, results in decreased microstressing. Further, inclusion of different vitamin C derivatives that have different affinity for water and fats can increase the bioavailability of vitamin C in the skin. The differing affinities provide different absorption pathways into the skin.

A lipophilic molecule has the ability to dissolve in fats, oils, lipids, and non-polar solvents. A hydrophilic molecule has the ability to dissolve in water. Thus, the lipophilic molecule has an affinity for fats, oils, lipids, and non-polar solvents, collectively referred to as “fats” for the sake of simplicity, and the hydrophilic molecule has an affinity for water. An amphiphilic molecule has a hydrophobic (non-polar) region and a hydrophilic (polar) region and can be soluble in water and in fats.

The outer stratum corneum is lipophilic and the intercellular channels and cytoplasm are hydrophilic. A hydrophilic active ingredient, such as the L-ascorbic acid form of vitamin C, is absorbed via the intercellular channels and cytoplasm. Thus, the conventional skincare formulations that only include L-ascorbic acid as an active ingredient have a single absorption pathway, limiting the bioavailability of vitamin C present in the skin. Aspects of the present disclosure recognizes that a selection of at least three active vitamin C derivatives that have different affinity for water and fats provides multiple absorption pathways, which can increase the bioavailability of vitamin C present in the skin. Exemplary skincare formulations disclosed herein should include at least one lipophilic active ingredient that can be absorbed through the outer stratum corneum, at least one hydrophilic active ingredient that can be absorbed through the intercellular channels and cytoplasm, and at least one amphiphilic active ingredient that is soluble in water and fats. The amphiphilic active ingredient is more readily absorbed through both the hydrophilic absorption pathway and the lipophilic absorption pathway than the hydrophilic active ingredient or the lipophilic active ingredient.

### Active Vitamin C Derivatives

Embodiments of the skincare formulation of the present disclosure include active vitamin C derivatives that are stable and can be converted to L-ascorbic acid, the active form of vitamin C in the skin, upon absorption by the skin. Further, the active vitamin C derivatives selected for inclusion in the exemplary skincare formulation have different affinity for water and fats to increase the bioavailability of the vitamin C. Thus, the active vitamin C derivatives include at least one hydrophilic active ingredient, at least one lipophilic active ingredient, and at least one amphiphilic active ingredient. In the exemplary formulations described herein, the active vitamin C derivatives exclude L-ascorbic acid, which is only stable in skincare formulations at a pH that causes undesirable microstressing of the skin and damage to the skin's microbiome, exclude anhydrous systems in which L-ascorbic acid is stable, due to the undesirable aesthetic application on the skin, and exclude ascorbyl palmitate, which is believed to promote skin damage as described in more detail below.

Examples of at least some active ingredients are discussed in more detail below.

### Bis-Glyceryl Ascorbate (BGA).

BGA is a stable vitamin C derivative produced by binding together ascorbic acid with two glyceryl groups. BGA is hydrophilic and non-ionic. BGA inhibits melanin production and provides moisture and strengthens the cornified cell envelope in the stratum corneum.

An effective amount of BGA (or one or more hydrophilic vitamin C derivatives that includes or excludes BGA) is less than about 10 wt % of the skincare formulation, or more specifically, between about 5-9 wt % of the skincare formulation. In a particular embodiment, the effective amount of the hydrophilic vitamin C derivative is about 7 wt % of the skincare formulation.

### Myristyl 3-Glyceryl Ascorbate (MGA).

MGA is a stable vitamin C derivative produced by binding together ascorbic acid, glycerin, and an alkyl group. MGA is amphiphilic and non-ionic and has oil-in-water emulsifying properties. MGA inhibits melanin production and has antibacterial properties. Other amphiphilic vitamin C derivatives may be included with MGA or substituted for MGA, excluding ascorbyl palmitate. At least one study has shown that ascorbyl palmitate promotes UV-B-induced lipid peroxidation, which may intensify the skin damage attributed to UV radiation.

An effective amount of MGA is limited by the amount of amphiphilic vitamin C that would cause the skincare formulation to develop the undesirable heavy or oily feel. Accordingly, an amount of MGA (or of one or more amphiphilic vitamin C derivatives that includes or excludes MGA) is less than about 10 wt % of the skincare formulation, or more specifically less than about 5 wt % of the skincare formulation. In a particular embodiment, the effective amount of an amphiphilic vitamin C derivative is between 0.5-2.5 wt % of the skincare formulation, or about 1 wt % of the skincare formulation. Other amphiphilic vitamin C derivatives substituted in place of MGA or included along with MGA may not impart the same heavy, oily feel and could be included in higher concentrations.

### Tetrahexyldecyl Ascorbate (THD Ascorbate)

THD Ascorbate is a stable, vitamin C derivative produced by adding hydrocarbon chains, e.g., trihexyl hexadecane, to ascorbic acid to impart stability and oil solubility. THD Ascorbate is lipophilic.

An effective amount of THD Ascorbate (or one or more lipophilic vitamin C derivatives that includes or excludes THD Ascorbate) is less than about 10 wt % of the skincare formulation, or more specifically, between about 5-9 wt % of the skincare formulation. In a particular embodiment, the effective amount of the lipophilic vitamin C derivative is about 7 wt % of the skincare formulation.

### Carrier Base

The active ingredients can be mixed into a carrier base to form an emulsion.

### Example Formulations

Example 1. A first exemplary formulation includes an effective amount of at least three active vitamin C derivatives, each of which can be converted into the L-ascorbic acid form of vitamin C upon absorption, and at least some of the active vitamin C derivatives having differing affinities for water and fats. The active vitamin C derivatives include a lipophilic vitamin C derivative, a hydrophilic vitamin C derivative, and an amphiphilic vitamin C derivative mixed into a carrier base to form an emulsion. The plurality of active vitamin C derivatives can be included in an effective amount that combines to form between 5-30 wt % of the skincare formulation, or more specifically, between 8-25 wt % of the skincare formulation. In a particular embodiment, the plurality of active vitamin C derivatives can be combined to form between 10-20 wt % of the skincare formulation. Additionally, a pH of the skincare formulation is between 3.5 and 7, or more specifically the pH is approximately skin neutral. In a first embodiment of this first formulation, the lipophilic vitamin C derivative and the hydrophilic vitamin C derivative are present in greater concentrations than the amphiphilic vitamin C derivative. In a second embodiment of this first formulation, the lipophilic vitamin C derivative and the hydrophilic vitamin C derivative are present in greater concentrations than the amphiphilic vitamin C derivative and are included in approximately equal amounts. In a third embodiment of this first formulation, the lipophilic vitamin C derivative and the hydrophilic vitamin C derivative are present in greater concentrations than the amphiphilic vitamin C derivative and are included in amounts that differ by less than 50%. In a fourth embodiment of this first formulation, none of the active vitamin C derivatives are present in an amount that is greater than 10 wt % of the skincare formulation. In a fifth embodiment of this first formulation, the plurality of active vitamin C derivatives are present in approximately equal amounts. In a sixth embodiment of this first formulation, the lipophilic vitamin C derivative is included in an amount between 3-15 wt % of the skincare formulation, or more specifically, between 4-12 wt % of the skincare formulation, or even more specifically, between 5-10 wt % of the skincare formulation; the hydrophilic vitamin C derivative is included in an amount between 3-15 wt % of the skincare formulation, or more specifically, between 4-12 wt % of the skincare formulation, or even more specifically, between 5-10 wt % of the skincare formulation; and the amphiphilic vitamin C derivative is included in an amount of about 0.10-10 wt % of the skincare formulation, or more specifically, between 0.25-8 wt % of the skincare formulation, or even more specifically, between 0.5-5 wt % of the skincare formulation. In a seventh embodiment of this first formulation, the lipophilic vitamin C derivative is included in an amount of about 7 wt % of the skincare formulation, the hydrophilic vitamin C derivative is included in an amount of about 7 wt % of the skincare formulation, and the amphiphilic vitamin C derivative is included in an amount of about 1 wt % of the skincare formulation.

Example 2. A second exemplary formulation includes an effective amount of active vitamin C derivatives, each of which can be converted into the L-ascorbic acid form of vitamin C upon absorption, and at least some of the active vitamin C derivatives having differing affinities for water and fats. In particular, the active vitamin C derivatives include BGA, MGA, and THD Ascorbate mixed into a carrier base to form an emulsion. The plurality of active vitamin C derivatives can be included in an effective amount that combines to form between 10.5-19.5 wt % of the skincare formulation, and a pH of the skincare formulation is between 3.5 and 7, or more specifically the pH is approximately skin neutral. In a first embodiment of this second formulation, BGA and THD Ascorbate are present in greater concentrations than MGA.

In a second embodiment of this second formulation, BGA and THD Ascorbate are present in greater concentrations than MGA and are included in approximately equal amounts. In a third embodiment of this second formulation, the BGA and THD Ascorbate are present in greater concentrations than the amphiphilic vitamin C derivative and the BGA and THD ascorbate are included in amounts that differ by less than 50%. In a fourth embodiment of this second formulation, none of the active vitamin C derivatives are present in an amount that is greater than 10 wt % of the skincare formulation. In a fifth embodiment of this second formulation, the BGA, MGA, and THD ascorbate are included in approximately equal amounts. In a sixth embodiment of this second formulation, the BGA is included in an amount between 5-9 wt % of the skincare formulation, THD Ascorbate is included in an amount between 5-9 wt % of the skincare formulation, and MGA is included in an amount between 0.5-2.5 wt % of the skincare formulation. In a seventh embodiment of this second formulation, the BGA is included in an amount of about 7 wt % of the skincare formulation, THD Ascorbate is included in an amount of about 7 wt % of the skincare formulation, and MGA is included in an amount of about 1 wt % of the skincare formulation.

Although embodiments of the invention have been described with reference to several elements, any element described in the embodiments described herein are exemplary and can be omitted, substituted, added, combined, or rearranged as applicable to form new embodiments. A skilled person, upon reading the present specification, would recognize that such additional embodiments are effectively disclosed herein. For example, where this disclosure describes characteristics, structure, size, shape, arrangement, concentration, or composition for an element or process for making or using an element or combination of elements, the characteristics, structure, size, shape, arrangement, concentration, or composition can also be incorporated into any other element or combination of elements, or process for making or using an element or combination of elements described herein to provide additional embodiments.

Additionally, where an embodiment is described herein as comprising some element or group of elements, additional embodiments can consist essentially of or consist of the element or group of elements. Also, although the open-ended term “comprises” is generally used herein, additional embodiments can be formed by substituting the terms “consisting essentially of” or “consisting of.”

While this invention has been particularly shown and described with reference to preferred embodiments, it will be understood by those skilled in the art that various changes in form and detail may be made therein without departing from the spirit and scope of the invention. The inventors expect skilled artisans to employ such variations as appropriate, and the inventors intend the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. A skincare formulation comprising:
a carrier base; and
an effective amount of active vitamin C derivatives mixed into the carrier base, wherein:
the active vitamin C derivatives include a hydrophilic vitamin C derivative, a lipophilic vitamin C derivative, and an amphiphilic vitamin C derivative,
the active vitamin C derivatives exclude L-ascorbic acid and ascorbyl palmitate, and
a pH of the skincare formulation is greater than 3.5.

2. The skincare formulation of claim 1, wherein the lipophilic vitamin C derivative and the hydrophilic vitamin C derivative are present in higher concentrations than the amphiphilic vitamin C derivative.

3. The skincare formulation of claim 2, wherein the lipophilic vitamin C derivative and the hydrophilic vitamin C derivative are included in approximately equal amounts.

4. The skincare formulation of claim 2, wherein the lipophilic vitamin C derivative and the hydrophilic vitamin C derivative are included in amounts that differ by less than 50%.

5. The skincare formulation of claim 1, wherein none of the active vitamin C derivatives are present in an amount that is greater than 10 wt % of the skincare formulation.

6. The skincare formulation of claim 1, wherein the lipophilic vitamin C derivative is included in an amount between 3-15 wt % of the skincare formulation, the hydrophilic vitamin C derivative is included in an amount between 3-15 wt % of the skincare formulation, and the amphiphilic vitamin C derivative is between 0.10-10 wt % of the skincare formulation.

7. The skincare formulation of claim 6, wherein the lipophilic vitamin C derivative is included in an amount between 4-12 wt % of the skincare formulation, the hydrophilic vitamin C derivative is included in an amount between 4-12 wt % of the skincare formulation, and the amphiphilic vitamin C derivative is between 0.25-8 wt % of the skincare formulation.

8. The skincare formulation of claim 7, wherein the lipophilic vitamin C derivative is included in an amount between 5-10 wt % of the skincare formulation, the hydrophilic vitamin C derivative is included in an amount between 5-10 wt % of the skincare formulation, and the amphiphilic vitamin C derivative is included in an amount of about 0.5-5 wt % of the skincare formulation.

9. The skincare formulation of claim 8, wherein the lipophilic vitamin C derivative is included in an amount of about 7 wt % of the skincare formulation, the hydrophilic vitamin C derivative is included in an amount of about 7 wt % of the skincare formulation, and the amphiphilic vitamin C derivative is included in an amount of about 1 wt % of the skincare formulation.

10. The skincare formulation of claim 1, wherein the effective amount of the active vitamin C derivates combines to form between 5-30 wt % of the skincare formulation.

11. The skincare formulation of claim 10, wherein the effective amount of the active vitamin C derivatives combines to form between 8-25 wt % of the skincare formulation.

12. The skincare formulation of claim 11, wherein the effective amount of the active vitamin C derivatives combines to form between 10-20 wt % of the skincare formulation.

13. The skincare formulation of claim 1, wherein the hydrophilic vitamin C derivative is Bis-Glyceryl Ascorbate.

14. The skincare formulation of claim 1, wherein the lipophilic vitamin C derivative is Tetrahexyldecyl Ascorbate.

15. The skincare formulation of claim 1, wherein the amphiphilic vitamin C derivative is Myristyl 3-Glyceryl Ascorbate.

16. The skincare formulation of claim 1, wherein the pH is less than 7.

17. The skincare formulation of claim 1, wherein the pH is skin neutral.

18. The skincare formulation of claim 1, wherein the carrier base and the active vitamin C derivatives form an emulsion.
